(19) United States
(12) Patent Application Publication
(10) Pub. No.: US 2016/0178647 A1
(43) Pub. Date: Jun. 23, 2016

(54) Gum Condition Assessment

(71) Applicant: The Procter & Gamble Company, Cincinnati, OH (US)

(72) Inventors: Jinlan Chang, Beijing (CN); Wendi Zhang, Beijing (CN); Yanyan He, Beijing (CN); Ying Tang, Beijing (CN)

(21) Appl. No.: 14/964,655

(22) Filed: Dec. 10, 2015

(30) Foreign Application Priority Data
Dec. 19, 2014 (CN) 2014/094346

(51) Int. Cl. G01N 33/72

(52) U.S. Cl. CPC G01N 33/721; G01N 2333/805; G01N 2800/18; G01N 2800/52

(57) ABSTRACT

A method of assessing a gum condition of an individual is provided.

### FIELD OF THE INVENTION

The invention is directed to methods of assessing a gum condition in a individual.

### BACKGROUND OF THE INVENTION

It is widely recognized by dental professionals that “gingival bleeding” or “gum bleeding” is a symptom of a potential gum condition. Dental professionals make this assessment when referring to bleeding that is induced by manipulating tissue with a periodontal probe at the depth of the gingival sulcus. There are many possible causes of gingival bleeding including formation and accumulation of plaque at the gum line. Some gum conditions, such as gingivitis, can be treated with proper brushing and flossing of teeth (i.e., good oral hygiene). However, chronic gingivitis can progress to periodontitis, which is an irreversible periodontal infection characterized by alveolar bone loss, and can eventually lead to tooth loss.

At least one report has over 70% of the Chinese population with varying degrees of gum disease. Furthermore, many individuals do not have access to dental professionals. Even with access to dental professions, there is a certain amount of subjectivity in assessing, for example, the relative status of a gum condition (e.g., mild, moderate, severe). One reported approach is by counting gum bleeding sites (induced with the periodontal probe). That is, typical measures of gum health or an improvement in a gum condition is by clinically observing a reduction in the number bleeding sites. However, the volume of bleeding is typically not taken into account. Although there may be a clinically evaluated reduction in the overall number of bleeding sites, the remaining (untreated) bleeding sites may still bleed at differing rates due to pocket depth, inflammation, and the like. There is a need to objectively quantifying the amount blood to provide a more objective measure of the relative status of a gum condition. Furthermore, objective measures such as quantifying blood volume, is more relevant and meaningful to the general public.

Accordingly, there is a need for a method to assess a gum condition of an individual, and to do so minimizing subjectivity and cost and while maximizing convenience. It is an objective to provide a measure this is more relevant and meaningful to the general public. This is particularly true in developing markets such as China.

### SUMMARY OF THE INVENTION

The present invention is based, in part, on the observation that hemoglobin contained in blood can be a proxy to assess a gum condition. In a nutshell, the more gingival bleeding there is, the more likely there is a gum condition (or the greater severity of the gum condition). Indeed hemoglobin that is contained in gingival bleeding can be quantified (and correlated to clinical assessments of gum conditions). The present invention is also based upon incorporating tooth brushing, a beneficial oral hygiene habit that can help in treating a gum condition, into the assessment method. Without wishing to be bound theory, such an approach reduces barriers for individuals to participate in the assessment, and re-enforces the need for good oral hygiene such as tooth brushing. In other words, the present invention incorporates a relevant oral hygiene activity into the gum condition assessment method.

The methods herein described can also provide a means to pre-screen individuals that many need further clinical evaluation.

The methods herein described can also provide a means to improve or assess oral care products or oral hygiene treatment regimens useful in the treatment of a gum condition.

A first aspect of the invention provides a method of assessing a gum condition of a subject individual comprising the steps: (a) tooth brushing within an oral cavity of the subject individual; (b) post-brushing rinsing the brushed oral cavity with a post-brushing rinse solution, wherein the post-brushing rinse solution comprises a post-brushing rinse solution volume from 1 ml to 50 ml, wherein the post-brushing rinsing is for a predetermined post-brushing rinsing time from 1 second to 1 minute; (c) collecting expectorated post-brushing rinse solution from the subject individual; (d) measuring hemoglobin quantity contained in the collected post-brushing rinse solution; and (e) comparing the measured hemoglobin quantity against a known standard of gum conditions corresponding to hemoglobin quantities to assesses the gum condition of the subject individual from the compared hemoglobin quantity.

A second aspect of the invention provides for a method of assessing a gum condition of a subject individual comprising the steps: (a) tooth brushing within an oral cavity of the subject individual; (b) post-brushing rinsing the brushed oral cavity with a post-brushing rinse solution, wherein the post-brushing rinse solution comprises a post-brushing rinse solution volume from 1 ml to 50 ml, wherein the post-brushing rinsing is for a predetermined post-brushing rinsing time from 1 second to 1 minute; (c) collecting expectorated post-brushing rinse solution from the subject individual; and (d) visually comparing the collected post-brushing rinse solution against a known standard of gum conditions corresponding to hemoglobin quantities to assess the gum condition of the subject individual.

A third aspect of the invention provides for a method of assessing effectiveness of a product or regimen in the treatment of a gum condition comprising the steps:

(a) assessing an individual's first gum condition comprising the steps:
(i) tooth brushing within an oral cavity of the individual;
(ii) post-brushing rinsing the brushed oral cavity with a first post-brushing rinse solution, wherein the post-brushing rinse solution comprises a post-brushing rinse solution volume from 1 ml to 50 ml, wherein the post-brushing rinsing is for a predetermined post-brushing rinsing time, wherein the predetermined post-brushing rinsing time is from 1 second to 1 minute;
(iii) collecting expectorated first post-brushing rinse solution from the individual;
(iv) measuring hemoglobin quantity contained in the first post-brushing rinse solution;
(v) comparing the first measured hemoglobin quantity against a known standard of gum conditions corresponding to hemoglobin quantities to assesses the first gum condition of the individual;
   (b) subjecting the individual to the product or regimen;
   (c) assessing the individual's second gum condition comprising the steps:
(i) tooth brushing within the oral cavity of the individual;
(ii) post-brushing rinsing the brushed oral cavity with a second post-brushing rinse solution having a post-brushing rinse solution volume, and for the predetermined post-brushing rinsing time;
(iii) collecting expectorated second post-brushing rinse solution from the individual;
(iv) measuring hemoglobin quantity contained in the second post-brushing rinse solution;
(v) comparing the second measured hemoglobin quantity against the known standard of gum conditions corresponding to hemoglobin quantities to assess the second gum condition of the individual; and
   (d) comparing the first gum condition assessment to the second gum condition assessment to assess the effectiveness of the product or regimen in the treatment of the gum condition in the individual.

A fourth aspect of the invention provides for a computer-aided system of assessing a gum condition of a subject individual comprising: (a) a sampling section for sampling expectorated post-brushing rinsing solution of the subject individual; (b) a measuring section in communication with the sampling section, wherein the measuring section is configured to measure hemoglobin quantity contained in the expectorated post-brushing rinsing solution; and (c) a computing section in communication with the measuring section, wherein the computing section is configured for receiving and statistically analyzing the measured hemoglobin to the gum condition.

These and other features, aspects, and advantages of the present invention will become evident to those skilled in the art from the detailed description which follows.

### DETAILED DESCRIPTION OF THE INVENTION

### Instructions

An optional, but preferred, initial step is to provide instructions to the individual regarding the methods described herein. Instructions may be provided by a live demonstration, pre-recorded video, literature (e.g., text and pictures), web-based, or combination thereof. Preferably the instructions comprise a video since users typically find it easier to model viewed behavior as compared to reading about it. A pre-recorded video is more cost effective than a live demonstration by an instructor. Specific mention is made to instructing the individual about brushing; preferably having the individual tooth brushing their own oral cavity. Alternatively, a trained tester brushes the individual's oral cavity. Other oral hygiene products and regimens may be optionally included. The instructions may also provide the added benefit of teaching or reinforcing proper tooth brushing techniques (or other oral hygiene habits).

### Pre-Rinsing

An optional, but preferred step after optionally providing instructions to the individual, is pre-rinsing the oral cavity of the individual, before the brushing step (described below), with a pre-rinse solution. Preferably the pre-rinse solution comprises a predetermined pre-rinse volume, more preferably the predetermined pre-rinse volume is from 1 ml to 5 ml, yet more preferably from 1 ml to 2.5 ml. Without wishing to be bound by theory, having less than 1 ml will leave the oral cavity too dry, even perhaps irritating the mouth. However, volumes greater than about 2.5 ml may lead to undesirably variability in testing. Another advantage of the pre-rinsing step is to potentially remove contaminants from the oral cavity that could negatively impact results.

Preferably the pre-rinsing step is for a predetermined pre-rinsing time, more preferably the predetermined pre-rinsing time is from 1 second to 1 minute, yet more preferably from 2 seconds to 10 seconds. Preferably the pre-rinse solution comprises from 90% to 100%, more preferably from 95% to 100%, yet more preferably 98% to 100% water by weight of the pre-rinse solution. The pre-rinse solution may comprise minor ingredients such as salt (to provide an isotonic saline solution) or contain antibacterial agents and the like. Preferably the pre-rinse solution is simply water. Indeed simply using water is cost effective.

### Immersing a Brush

An optional, but preferred step after optionally pre-rinsing the oral cavity of the individual, but before the brushing step (described below), comprises the step of immersing a brush in a brush immersing solution. Preferably the immersing solution comprises from 90% to 100%, more preferably from 95% to 100%, yet more preferably 98% to 100% water by weight of the immersing solution. The brush immersing solution may comprise minor ingredients such as salt (to provide an isotonic saline solution) or contain antibacterial agents and the like. More preferably the immersing solution is simply water. Indeed simply using water is cost effective.

Preferably the step of immersing the brush is for a predetermined immersing time, preferably the predetermined immersing time is from 0.1 second to 5 seconds, more preferably from 0.5 seconds to 2 seconds, alternatively about 1 second.

### Brushing

A step in the present invention is tooth brushing within an oral cavity of the individual. The individual may conduct the tooth brushing or a trained tester may conduct the tooth brushing to the individual.

Preferably the tooth brushing further comprises tooth brushing for a predetermined brushing time. Preferably the predetermined brushing time is from 30 seconds to 5 minutes, more preferably from 1 minute to 3 minutes, alternatively for about 2 minutes. The tooth brushing may by way of a manual toothbrush or an automated toothbrush (e.g., an electric toothbrush, gum brush (e.g., Gum Health™)). For purposes of clarification, the term “tooth brushing” should be construed broadly to include those brushes designed to brush gums. The portion of the oral cavity that is brushed may include a tooth or gum, or combination thereof. In one non-limiting example, the interface between the tooth and gum is brushed.

### Post-Brushing Rinsing

A step in the present invention is post-brushing rinsing the oral cavity with a post-brushing rinse solution. Best results are typically obtained when the post-brushing rinsing step is conducted immediately after the aforementioned tooth brushing step. Preferably the post-brushing rinse solution comprises a predetermined post-brushing rinse solution volume. Preferably the predetermined post-brushing rinse solution volume is from 1 ml to 50 ml, more preferably from 5 ml to 40 ml, yet more preferably from 5 ml to 15 ml, alternatively about 10 ml. Without wishing to be bound by theory, if the volume is less than 5 ml, it may lead to undesirably variability in testing. However, volumes greater than 50 ml may provide for a hemoglobin concentration that is too low to be accurately detected by some methods (e.g., Ultra Violet—Visible Light Analysis described in further detail below). More than one post-brushing rinse can be employed.

Preferably the post-brushing rinsing step is for a predetermined post-brushing rinsing time. More preferably the predetermined post-brushing rinsing time is from 1 second to 1 minute, yet more preferably from 5 seconds to 15 seconds, alternatively about 10 seconds. Preferably the post-brushing rinse solution comprises from 90% to 100%, more preferably from 95% to 100%, yet more preferably 98% to 100% water by weight of the pre-rinse solution. The post-brushing rinse solution may comprise minor ingredients such as salt (to provide an isotonic saline solution) or contain antibacterial agents and the like. Preferably the post-brushing rinse solution is simply water. Indeed simply using water is cost effective.

It is particularly preferred the step of collecting the saliva sample from the brushed oral cavity of the individual comprising collecting the post-brushing rinse solution.

### Collection

A step in the present invention is collecting expectorated post-brushing rinse solution from the individual. This can be way of a cup, test tube, vial, or the like.

### Centrifugation

An optional, but preferred step after the aforementioned collection of expectorated post-brushing rinse solution is centrifuging the collected saliva sample before measuring hemoglobin quantity (described below). Preferably the centrifuging step is for a predetermined centrifuging time, more preferably the predetermined centrifuging time is from 1 minute to 20 minutes, yet more preferably from 5 minutes to 15 minutes. Preferably the centrifuging step is spun at a speed as to reach at centrifugal force greater than 30 gravitational force (g-force), preferably the centrifugal force is between 30 g-force to 25,000 g-force, preferably from 8,000 g-force to 12,000 g-force, alternatively about 10,733 g-force. Without wishing to be bound by theory, centrifuging the collected saliva sample removes solids or other ingredients that may interfere with measuring hemoglobin quantity.

### Measuring Hemoglobin Quantity

A step in present invention is measuring hemoglobin quantity contained in the collected post-brushing rinse solution (or centrifuged post-brushing rinse solution). Any known method of measuring hemoglobin quantity may be used including, but not limited to, colormetric, spectrophotometric, chemical indicator, ELISA, colloidal gold techniques, and other immunological techniques, and the like.

Those tests that are simple, quick, and inexpensive are preferred. One especially preferred method of measuring hemoglobin quantity contained in the collected saliva sample is Ultra Violet—Visible Light Analysis (“UVVLA”), more preferably wherein the UVVLA is conducted at a wavelength from 340 nm to 440 nm, preferably from 400 nm to 410 nm, alternatively about 406 nm.

Comparing against Known Standard

A step in present invention is comparing the measured hemoglobin quantity against a known standard of gum conditions corresponding to hemoglobin quantities. Such standards are commercially available. Of course the known standard will depend upon the hemoglobin quantity measuring test employed.

### Visually Comparing

A step in the present invention is visually comparing the collected post-brushing rinse solution against a known standard of gum conditions corresponding to hemoglobin quantities. When analytical materials or instruments are not available or less accurate or precise results are acceptable, the collected post-brushing rinse solution may simple be visually compared, i.e., with an unaided eye, to physical standards, pictures, photo, video or the like.

### Communicating Gum Health

An optional, but preferred step in the methods of the present invention, is communicating the assessed gum health. This tool, much like the initial optional instructions, can be provided with a live consultation, pre-recorded video, literature (e.g., text and pictures), web-based, or combinations thereof. The communication is preferably tailored to the specific gum health status assessed, most even more preferably contain information what can and should be done by the individual to remedy the individual's gum condition if an undesirable gum condition is assessed. Obtaining and showcasing an endorsement or certification from a well recognized and respected industry group may also be helpful in adoption of the methods described herein.

### Assessing Effectiveness of a Product or Regimen

Another aspect of the invention provides for a method of assessing effectiveness of a product or regimen in the treatment of a gum condition of an individual. The method comprising the steps: firstly assessing an individual's first gum condition comprising the sub-steps: (i) tooth brushing within an oral cavity of the individual; (ii) post-brushing rinsing the brushed oral cavity with a first post-brushing rinse solution for a predetermined post-brushing rinsing time, wherein the predetermined post-brushing rinsing time is from 1 second to 1 minute; (iii) collecting expectorated first post-brushing rinse solution from the individual; (iv) measuring hemoglobin quantity contained in the first post-brushing rinse solution; and (v) comparing the first measured hemoglobin quantity against a known standard of gum conditions corresponding to hemoglobin quantities to assesses the first gum condition of the individual.

A second step is subjecting the individual to the product or regimen. The product or regimen may comprise a toothbrush, toothpaste, mouth rinse, floss, teeth whitening strips, or combinations thereof. Indeed new ingredients or products may be designed or tested.

A third step is assessing the individual's second gum condition (after the individual is subjected to the product or regimen) comprising the sub-steps: (i) tooth brushing within the oral cavity of the individual; (ii) post-brushing rinsing the brushed oral cavity with a second post-brushing rinse solution for the predetermined post-brushing rinsing time; (iii) collecting expectorated second post-brushing rinse solution from the individual; (iv) measuring hemoglobin quantity contained in the second post-brushing rinse solution; and (v) comparing the second measured hemoglobin quantity against the known standard of gum conditions corresponding to hemoglobin quantities to assess the second gum condition of the individual.

A fourth step is comparing the first gum condition assessment to the second gum condition assessment to assess the effectiveness of the product or regimen in the treatment of the gum condition in the individual.

Preferred aspects of the methods include those as previously described for the earlier aspects of the invention (that are not repeated here for the sake of brevity).

### Computer Aided System

Another aspect of the invention provides for a computer-aided system of assessing a gum condition of a subject individual comprising: (a) a sampling section for sampling expectorated post-brushing rinsing solution of the subject individual; (b) a measuring section in communication with the sampling section, wherein the measuring section is configured to measure hemoglobin quantity contained in the expectorated post-brushing rinsing solution; (c) a computing section in communication with the measuring section, wherein the computing section is configured for receiving and statistically analyzing the measured hemoglobin to a gum condition.

Preferably the computing section comprises: (i) an input module in communication with the measuring section, wherein the input module is for inputting input data, wherein the input data comprises demographic information of the subject individual or medical information of the subject individual; and (ii) a data processing module in communication with the input module, wherein the data processing module is configured for statistically analyzing the input data to correlate with a statistical significance to the gum condition.

Preferably the step of measuring hemoglobin quantity contained in the saliva sample is Ultra Violet—Visible Light Analysis (“UVVLA”) as described earlier.

### EXAMPLES

### Clinical Study

The detection and quantification of gingival hemoglobin, using the inventive method of the present invention, in a clinical study is conducted.

### Overall Study Design and Plan is Described

The study is a two-leg, 3 days clinical study. Around 40 qualified subjects meeting all the study entrance criteria are enrolled into the study. Gingivitis is measured by hemoglobin sampled at baseline and at day 3.

On the first day of baseline visit, subjects receive power brush sampling for gingival hemoglobin. Then all the subjects are given one tube of Crest® Regular Caries Protection toothpaste and one Crest® MeiLiLiangJie toothbrush and are asked to use in their normal oral hygiene habits for the duration of study. At the second day, subjects receive manual brush sampling for gingival hemoglobin. After sampling, the subjects are randomized to two treatment groups: Prophy and Non-prophy. The subjects who are assigned to Prophy group receive dental prophylaxis.

At the first day of day 3 visit: the subjects receive power tooth brush sampling for gingival hemoglobin. At the second day, the subjects receive manual tooth brush sampling. Specific procedures are listed below (Table 1).

TABLE 1
Study Schedule by Procedure Type and Visit

| | BASELINE | | DAY 3 | |
|---|---|---|---|---|
| | VISIT | | VISIT | |
| PROCEDURE: | Day 1 | Day 2 | Day 1 | Day 2 |
| INFORMED CONSENT | X | | | |
| MEDICAL HISTORY | X | | | |
| DEMOGRAPHICS | X | | | |
| INCLUSION/EXCLUSION | X | | | |
| CONTINUANCE CRITERIA | | X | X | X |
| HEMOGLOBIN SAMPLING | X | X | X | X |
| METHOD | | | | |
| DENTAL PROPHYLAXIS | | X | | |
| ADVERSE EVENT | | X | X | X |
| RECORDING | | | | |
| SUBJECT ACCOUNTABILITY | | | | X |

Baseline Visit: At the first day, subjects are asked to read and sign an informed consent and receive a signed copy for their records. Personal medical history information is obtained and reviewed and retained as site source documentation. Demographic information and inclusion criteria are obtained and documented on the appropriate case report form (CRF). Then subjects receive power brush sampling for gingival hemoglobin. Then all the subjects are given one tube of CREST® Regular Caries Protection toothpaste (Lot #40671864AA) and one CREST® MeiLiLiangJie toothbrush (Lot #OM003). Subjects are asked to use the assigned product during the course of the study and are reminded to refrain from smoking and any oral hygiene after 11 μm the night before and morning of their next visit and refrain from consuming anything by mouth (except a small amount of water) at least 4 hours prior to their next visit. General comments, if applicable, are recorded in the appropriate CRF.

At the second day, continuance criteria is assessed and documented on the appropriate CRF. Subjects receive manual brush sampling. Then the subjects are randomized to two treatment groups based on Ultra Violet—Visible Light Analysis (“UVVLA”) score of power brush, age, gender and smoking status: prophylaxis and non prophylaxis. The subjects who are assigned to Prophy group receive dental prophylaxis. Subjects are asked to use the assigned product during the course of the study and are reminded to refrain from smoking and any oral hygiene after 11pm the night before and morning of their next visit and refrain from consuming anything by mouth (except a small amount of water) at least 4 hours prior to their next visit. General comments, if applicable, are recorded in the appropriate CRF.

Day 3 Visit:

At the first day, continuance criteria is assessed and documented on the appropriate CRF. Then the subjects receive power brush sampling. Subjects are asked to use the assigned product during the course of the study and reminded to refrain from smoking and any oral hygiene after 11 μm the night before and morning of their next visit and refrain from consuming anything by mouth (except a small amount of water) at least 4 hours prior to their next visit. General comments, if applicable, are recorded in the appropriate CRF.

At the second day, continuance criteria is assessed and documented on the appropriate CRF. Subjects receive manual brush sampling. After completion of all procedures, final subject accountability is conducted, and the subjects are terminated from the study after receiving compensation.

### Inclusion Criteria:

In order to be included in the study, each subject must:

(i) be at least 18 years of age; (ii) provide written informed consent prior to participation and be given a signed copy of the informed consent form; (iii) possess a minimum of 12 natural anterior teeth; (iv) have gingivitis but without periodontitis; (v) be in good general health as determined by the Investigator/designee based on a review of the medical history/update for participation in the study; and (vi) a normal healthy adult.

Subjects reporting for their visit refrain from: performing any oral hygiene prior to their appointment; and consuming anything by mouth (except a small amount of water) at least 4 hours prior to their next visit. Subjects agree to receive dental prophylaxis required by study. Subjects self-report if there is visible bleeding when brushing in past 2 weeks. No periodontal disease and other oral diseases except tartar.

### Exclusion Criteria:

Subjects are excluded from study participation where there is evidence of any one the following: (i) periodontal disease beyond gingivitis (as noted above in inclusion criteria); (ii) teeth that are carious, fully-crowned, extensively restored and have heavy dental calculus; (iii) teeth with unresolved carious lesions; (iv) any condition which requires antibiotic premedication for the administration of a dental prophylaxis; (v) self-reported pregnancy or intent to become pregnant during the course of the study and nursing females; (vi) atypical discoloration or pigmentation in the gingival tissue; (vii) fixed facial orthodontic appliances; (viii) use of antibiotics any time during the study; (ix) any diseases or conditions that could be expected to interfere with the subject safely completing the study; and (x) inability to undergo imaging or other study procedures.

### Continuance Criteria:

Subjects may be excluded from the study or the analysis due to any one of the following: (i) use of antibiotics any time during the study; (ii) pregnancy or development an intercurrent medical condition which puts the subject at increased risk or invalidates the results of the study; (iii) use of any oral care products other than assigned study products; (iv) subject reporting for their visit having performed any oral hygiene prior to their appointment; (v) subjects reporting for their visit having consumed anything by mouth (except a small amount of water) at least 4 hours prior to their next visit; and (vi) UVVLA score >0.2.

### Oral Care Product(s):

No investigational product is used in this study. Subjects used CREST® Regular Caries Protection toothpaste and CREST® MeiLingLinagJie toothbrush in their normal habits during the study (per the Lot numbers previously described).

### Method of Assigning Subjects to Treatment Sequences:

To ensure sampling method randomization, subjects are randomized to two methods according to their visit order. To ensure treatment randomization, at the baseline visit, subjects are stratified based on the power brush UVVLA scores, age, gender, and smoking status within each cohort. Subjects are randomly assigned to two cohorts (Prophylaxis/Non-prophylaxis). Subjects who reside in the same household are assigned to the same group.

### Primary Efficacy Variable(s):

RGB Digital Imaging for sample picture taking: This system consists of a S2 Pro CCD high resolution digital camera manufactured by Fuji or a similar camera. It is equipped with a Nikon Micro Nikkor lens with a linear polarizer to permit cross-polarized light or similar optical system. Dedo lights, mounted to an imaging rig, provides lighting.

Prior to daily use, the system is calibrated to assure proper operation. Additionally, a color standard is centered and imaged every hour, and then is removed prior to imaging samples. For each examination period, the room is dark. The sample tube is placed on the chin rest, and a white balance is placed behind the sample tube. Operator follows standard capture image process and capture sample imaging.

Gingival hemoglobin method sampling procedure sampling and storage: Sampling method “1” by manual brush brushing and sample method “2” by Power brush brushing, both follow the same procedure listed as follows: (1) Watch the video of brush instruction; (2) Take an empty tube; (3) Take 2 ml water to rinse the whole mouth for 5 seconds; (4) Open the toothbrush from its packaging and put the whole brush head into 30 ml water for 1 second; (5) Brush whole teeth thoroughly for 2 minutes by oneself, expectorate in the tube; (6) After brushing, immediately rinse with 10 ml water for 10 seconds, expectorate in the tube; (7) Label the sample; (8) Centrifuge the sample at 10,733 g-force for 10 minutes; (9) Take 1 ml into a 2 ml tube, for UVVLA and sample picture taking immediately.

Ultraviolet—visible spectroscopy method (UVVLA): is used to find the specific absorbance of wavelength for hemoglobin, which is used for samples measurement. The absorbance of a sample is proportional to the concentration of Hemoglobin.

Determination of Sample Size: The sample size is based on logistical considerations and not power implications. Approximate 40 qualified subjects are enrolled into the study.

### Hypothesis:

The following hypothesis is tested separately each sample method (method 1 and 2) of UVVLA at Day 3:

Null: The mean change from baseline score is equal to 0.

Alternative: The mean change from baseline score is not equal to 0.

The following hypothesis are tested separately between groups (Prophy/Non-prophy) for each sample method of UVVLA at Day 3:

Null: The mean score for the Prophy group is equal to the mean score of the Non-prophy group.

Alternative: The mean score for the Prophy group is not equal to the mean score of the Non-prophy group.

The following hypothesis is tested of end point at Day 3:

Null: The mean change from baseline score is equal to 0.

Alternative: The mean change from baseline score is not equal to 0.

### Statistical and Analytical Plans:

Change from baseline is investigated using paired t-tests. Analysis of covariance is used to compare groups (Prophy/Non-prophy) with respect to each efficacy variable: UVVLA. A separate statistical model is used to test group difference at each visit. The baseline value of each respective endpoint is used as a covariate in each model. Statistical comparisons are two-sided with a 0.05 significance level.

### Overall Results of the Study are Provided

Statistical Methods: The minimum detection level for UVVLA is 20. If the score is less than 20, the level is changed to 10 as a final score. Log 10 transformations are applied to UVVLA variables to better meet the normal distribution hypothesis. The change from baseline is investigated using paired t-tests. Analysis of covariance is used to compare groups (Prophy/Non-prophy) with the efficacy variable of UVVLA. A separate statistical model is used to test group difference at each visit. The baseline value of UVVLA endpoint is used as a covariate in each model. Statistical comparisons are two-sided with a 0.05 significance level.

Study Population: Referring to Table 1 below, the study population consists of forty-two (42) subjects enrolled at the first visit and forty-two (42) subjects completing the study. Subjects' age ranged from 20 to 53 years with a mean of 33.74 and standard deviation of 7.75 years. Thirty-five (83%) of the subjects are female.

TABLE 1
Demographics Summary; All Subjects Treated

| | | | | |
|---|---|---|---|---|
| Demographic/ | | | | |
| Statistic or | Non-Prophy | Prophy | Overall | |
| Category | (n = 21) | (n = 21) | (n = 42) | P-value |

Age (Years)

| | | | | |
|---|---|---|---|---|
| Mean (SD) | 33.19 (7.33) | 34.29 (8.28) | 33.74 (7.75) | 0.6525^{a} |
| Min.-Max. | 24-46 | 20-53 | 20-53 | |

Sex

| | | | | |
|---|---|---|---|---|
| Female^{b} | 18 (86%) | 17 (81%) | 35 (83%) | 1.0000^{d} |
| Male^{b} | 3 (14%) | 4 (19%) | 7 (17%) | |

Smoking

| | | | | |
|---|---|---|---|---|
| N^{b} | 20 (95%) | 20 (95%) | 40 (95%) | 1.0000^{d} |
| Y^{b} | 1 (5%) | 1 (5%) | 2 (5%) | |

^{a}Two-sided Analysis of Variance for (ANOVA) p-value for the treatment comparison.
^{b}The number (percent) of subjects in each category.
^{c}Two-sided chi-square p-value for the treatment comparison.
^{d}Two-sided p-value for the treatment comparison from a Kruskal Wallis test.

### Efficacy Data

Manual brush: Referring to Table 2, at Baseline visit, Non-Prophy group and Prophy group starts from a Mean (SD) value of 1.93 (0.56) and 2.05 (0.49), respectively. At Day 3 visit, for Non-Prophy group and Prophy group, the Mean (SD) value is 1.80 (0.57) and 1.61 (0.51), respectively.

Referring to Table 3, the Non-Prophy group exhibits a difference from baseline relative to Prophy group with change from baseline means of 0.13 (0.36) (P=0.1162) and 0.44 (0.48) (P=0.0005). The Prophy group exhibits significantly lower (P=0.0325) mean by 15.0% relative to Non-Prophy group.

Power brush: Referring to Table 2, at Baseline visit, Non-Prophy group and Prophy group starts from a Mean (SD) value of 2.29 (0.50) and 2.31 (0.37), respectively. At Day 3 visit, for Non-Prophy group and Prophy group, the Mean (SD) value is 2.07 (0.55) and 1.78 (0.51), respectively.

TABLE 2
Hemoglobin—Day 03

| | | | | |
|---|---|---|---|---|
| Treatment | N | Mean (SD) | Median | Min.-Max. |

Manual—Baseline (overall treatment difference p-value: 0.4631)

| | | | | |
|---|---|---|---|---|
| Non-Prophy | 21 | 1.93 (0.56) | 2.01 | 1.00 to 2.79 |
| Prophy | 21 | 2.05 (0.49) | 2.06 | 1.00 to 3.03 |
| Overall | 42 | 1.99 (0.52) | 2.02 | 1.00 to 3.03 |

Manual—Day 03

| | | | | |
|---|---|---|---|---|
| Non-Prophy | 21 | 1.80 (0.57) | 1.84 | 1.00 to 2.83 |
| Prophy | 21 | 1.61 (0.51) | 1.65 | 1.00 to 2.64 |

Power—Baseline (overall treatment difference p-value: 0.8927)

| | | | | |
|---|---|---|---|---|
| Non-Prophy | 21 | 2.29 (0.50) | 2.21 | 1.65 to 3.19 |
| Prophy | 21 | 2.31 (0.37) | 2.25 | 1.68 to 3.01 |
| Overall | 42 | 2.30 (0.43) | 2.25 | 1.65 to 3.19 |

Power—Day 03

| | | | | |
|---|---|---|---|---|
| Non-Prophy | 21 | 2.07 (0.55) | 2.21 | 1.00 to 3.26 |
| Prophy | 21 | 1.78 (0.51) | 1.71 | 1.00 to 3.10 |

Referring to Table 3, the Non-Prophy group exhibited difference from baseline relative to Prophy group with changed from baseline means of 0.21 (0.49) (P=0.0606) and 0.53 (0.45) (P<0.0001). Prophy group exhibited significantly lower (P=0.0341) mean by 14.7% relative to Non-Prophy group.

TABLE 3
Change from Baseline; Hemoglobin—Day 03

| | | | | |
|---|---|---|---|---|
| Treatment | Mean (SD) | P-value^{a} | Median | Min.-Max. |

Manual—Day 03

| | | | | |
|---|---|---|---|---|
| Non-Prophy | −0.13 (0.36) | 0.1162 | −0.11 | −0.96 to 0.47 |
| Prophy | −0.44 (0.48) | 0.0005 | −0.39 | −1.47 to 0.58 |

Power—Day 03

| | | | | |
|---|---|---|---|---|
| Non-Prophy | −0.21 (0.49) | 0.0606 | −0.10 | −1.21 to 0.56 |
| Prophy | −0.53 (0.45) | <0.0001 | −0.58 | −1.39 to 0.22 |

^{a}Tests mean change from baseline versus 0 (2-sided paired-difference t-test)

Lastly, Table 4 below summarizes the efficacy results of the study. For the Powerbrush, the Prophy group exhibited significantly lower (P=0.0341) mean by 14.7% relative to Non-Prophy group. For the Manual brush, the Prophy group exhibited significantly lower (P=0.0325) mean by 15.0% relative to Non-Prophy group. This method is validated in Prophy vs. Non-prophy study (which is the best accepted standard in oral care industry and accordingly is a positive control).

TABLE 4
Efficacy Results
Analysis of Covariance: Adjusting for Baseline Score
Hemoglobin—Day03

| | | | | |
|---|---|---|---|---|
| | | | % Reduction | |
| | | | from Non- | P-Value^{b} |
| Treatment | N | Adj. Mean (SE) | Prophy^{a} | Prophy |

Manual (error variance = 0.161, baseline mean = 1.99)

| | | | | |
|---|---|---|---|---|
| Non-Prophy | 21 | 1.844 (0.088) | | 0.0325 |
| Prophy | 21 | 1.568 (0.088) | 15.0% | |

Power (error variance = 0.204, baseline mean = 2.30)

| | | | | |
|---|---|---|---|---|
| Non-Prophy | 21 | 2.081 (0.099) | | 0.0341 |
| Prophy | 21 | 1.774 (0.099) | 14.7% | |

^{a}Percent Change versus Control = 100 × (( Control − Experimental )/Control).
^{b}2-sided p-value comparing treatments using analysis of covariance.

Sample pictures of the clinical study are evaluated by visual perception study. The details are provided below:

### Study Design

9 healthy female subjects aged 18-35 years old in Beijing as a naïve grader joined the study.

The variable of deeper color is analyzed. The range of the score comparing pre and post treatment images is −4 to 4. The Prophy group treatment is dental prophylaxis; and the Non-Prophy group treatment, is the normal oral hygiene habit group.

The average score of all graders are calculated for each set of images (pre & post images). A simple t-test method is applied to test the score to assess if there is a significant change after treatment.

Treatment is considered statistically significantly better after applying treatment if the p-value is less than or equal to 0.05 (2-sided) and the average is negative.

### Results

A lower score means less redness after day 3.

Manual brush: In the Non-prophy group, the average mean is −0.85; in the Prophy group, the average mean is −1.51; the average means of both groups are statistically significantly lower than the hypothesis average mean 0 with P<0.039 and 0.001, respectively. Comparing the 2 groups, no statistically significant difference is observed with P=0.225;

Power brush: In the Non-prophy group, the average mean is −0.531; in Prophy group, the average mean is −2.05; the average mean of Prophy group is statistically significantly lower than the hypothesis average mean 0 with P<0.0001, the average mean of Non-prophy group does not show statistically significant lower than the hypothesis average mean 0. Comparing the 2 groups, statistically significant difference is observed with P=0.018.

TABLE 5
T-Test: Mean = 0

| Sampling | | | | Confidence |
|---|---|---|---|---|
| Method | Treatment | Mean (SD) | P-Value^{a} | Interval |
| Manual | Non- | −0.85 | 0.039 | (−1.644, −0.049) |
| | Prophy | (1.752) | | |
| Manual | Prophy | −1.51 | 0.001 | (−2.313, −0.714) |
| | | (1.757) | | |
| Power | Non- | −0.53 | 0.320 | (−1.611, 0.552) |
| | Prophy | (2.376) | | |
| Power | Prophy | −2.05 | <0.0001 | (−2.740, −1.356) |
| | | (1.520) | | |

^{a}T-Test P-Value against the hypothesis that the average score is Zero.

TABLE 6
T-Test: Comparing Treatment Mean

| Sampling | | | | | Confidence |
|---|---|---|---|---|---|
| Method | Treatment | Mean (SD) | Diff | P-Value | Interval |
| Manual | Non- | −0.85 | 0.6667 | 0.2254^{a} | (−0.428, 1.761)^{a} |
| | Prophy | (1.752) | | | |
| Manual | Prophy | −1.51 | | | |
| | | (1.757) | | | |
| Power | Non- | −0.53 | 1.5185 | 0.0180^{a} | (0.275, 2.763)^{a} |
| | Prophy | (2.376) | | | |
| Power | Prophy | −2.05 | | | |
| | | (1.520) | | | |

^{a}T-Test in the Pooled method for Equal Variance between groups.
^{b}T-Test in the Scatterthwaite method for Unequal Variance between groups.

### Conclusion

Power brush: Prophy group shows significantly better improvement after treatment and it exhibits significantly better change (P=0.018) relative to Non-prophy group. Manual brush: Both Prophy group and Non-prophy group show the significantly better improvement after treatment, but Prophy group is not significantly better (P=0.225) than the Non-prophy group.

This sampling method by power brush is validated in Prophy vs. Non-prophy study (which is best accepted standard in oral care industry and accordingly is a positive control). The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of assessing a gum condition of a subject individual comprising the steps:
(a) tooth brushing within an oral cavity of the subject individual;
(b) post-brushing rinsing the brushed oral cavity with a post-brushing rinse solution comprising a post-brushing rinse solution volume from 1 ml to 50 ml, for a predetermined post-brushing rinsing time, wherein the predetermined post-brushing rinsing time is from 1 second to 1 minute;
(c) collecting expectorated post-brushing rinse solution from the subject individual;
(d) measuring hemoglobin quantity contained in the collected post-brushing rinse solution; and
(e) comparing the measured hemoglobin quantity against a known standard of gum conditions corresponding to hemoglobin quantities to assesses the gum condition of the subject individual from the compared hemoglobin quantity.

3. The method of claim 1, wherein the predetermined post-brushing rinsing time is from 5 seconds to 15 seconds.

4. The method of claim 1, wherein the tooth brushing further comprising:
tooth brushing for a predetermined brushing time, wherein the predetermined brushing time is from 30 seconds to 5 minutes; and
wherein the oral cavity comprises a tooth, a gum, or combinations thereof.

5. The method of claim 1, further comprising a step of pre-rinsing the oral cavity, before the brushing step, with a pre-rinse solution, wherein the pre-rinsing step is for a predetermined pre-rinsing time, wherein the predetermined pre-rinsing time is from 1 second to 1 minute.

6. The method of claim 5, wherein the print-rinse solution having a pre-rinse volume from 1 ml to 5 ml.

7. The method of claim 1, wherein the post-brushing rinse solution and the pre-rinse solution are water.

8. The method of claim 1, further comprising the step of immersing a toothbrush in a brush immersing solution before the brushing step; and wherein the step of immersing the brush is for a predetermined immersing time, wherein the predetermined immersing time is from 0.1 second to 5 seconds.

9. The method of claim 1, further comprising the step of centrifuging the collected expectorated post-brushing rinse solution.

10. The method of claim 9, wherein the centrifuging step is for a predetermined centrifuging time, wherein the predetermined centrifuging time is from 1 minute to 20 minutes; and wherein the centrifuging step is conducted at a centrifugal force is between 30 gravitational-force to 25,000 gravitational-force.

11. The method of any one of claims 1, wherein the step of measuring hemoglobin quantity contained in the collected expectorated post-brushing rinse solution is by Ultra Violet—Visible Light Analysis (“UVVLA”).

12. A method of assessing a gum condition of a subject individual comprising the steps:
(a) tooth brushing within an oral cavity of the subject individual;
(b) post-brushing rinsing the brushed oral cavity with a post-brushing rinse solution comprising a post-brushing rinse solution volume from 1 ml to 50 ml, for a predetermined post-brushing rinsing time, wherein the predetermined post-brushing rinsing time is from 1 second to 1 minute;
(c) collecting expectorated post-brushing rinse solution from the subject individual; and
(d) visually comparing the collected post-brushing rinse solution against a known standard of gum conditions corresponding to hemoglobin quantities to assess the gum condition of the subject individual.

13. The method of claim 12, wherein the predetermined post-brushing rinsing time is from 5 seconds to 15 seconds.

14. The method of claim 12, wherein the tooth brushing further comprising:
tooth brushing for a predetermined brushing time, wherein the predetermined brushing time is from 30 seconds to 5 minutes; and
wherein the oral cavity comprises a tooth, a gum, or combinations thereof.

15. The method of claim 12, further comprising a step of pre-rinsing the oral cavity, before the brushing step, with a pre-rinse solution, wherein the pre-rinsing step is for a predetermined pre-rinsing time, wherein the predetermined pre-rinsing time is from 1 second to 1 minute.

16. The method of claim 15, wherein the print-rinse solution having a pre-rinse volume from 1 ml to 5 ml.

17. The method of claim 12, further comprising the step of immersing a toothbrush in a brush immersing solution before the brushing step; and wherein the step of immersing the brush is for a predetermined immersing time, wherein the predetermined immersing time is from 0.1 second to 5 seconds.

18. The method of claim 12, further comprising the step of centrifuging the collected expectorated post-brushing rinse solution.

19. The method of claim 18, wherein the centrifuging step is for a predetermined centrifuging time, wherein the predetermined centrifuging time is from 1 minute to 20 minutes; and wherein the centrifuging step is conducted at a centrifugal force is between 30 gravitational-force to 25,000 gravitational-force.

20. A method of assessing effectiveness of a product or regimen in the treatment of a gum condition comprising the steps:
(a) assessing an individual's first gum condition comprising the steps:
(i) tooth brushing within an oral cavity of the individual;
(ii) post-brushing rinsing the brushed oral cavity with a first post-brushing rinse solution comprising a post-brushing rinse solution volume from 1 ml to 50 ml, for a predetermined post-brushing rinsing time from 1 second to 1 minute;
(iii) collecting expectorated first post-brushing rinse solution from the individual;
(iv) measuring hemoglobin quantity contained in the first post-brushing rinse solution;
(v) comparing the first measured hemoglobin quantity against a known standard of gum conditions corresponding to hemoglobin quantities to assesses the first gum condition of the individual;
(b) subjecting the individual to the product or regimen;
(c) assessing the individual's second gum condition comprising the steps:
(i) tooth brushing within the oral cavity of the individual;
(ii) post-brushing rinsing the brushed oral cavity with a second post-brushing rinse solution with the post-brushing rinse solution volume and for the predetermined post-brushing rinsing time;
(iii) collecting expectorated second post-brushing rinse solution from the individual;
(iv) measuring hemoglobin quantity contained in the second post-brushing rinse solution;
(v) comparing the second measured hemoglobin quantity against the known standard of gum conditions corresponding to hemoglobin quantities to assess the second gum condition of the individual; and
(d) comparing the first gum condition assessment to the second gum condition assessment to assess the effectiveness of the product or regimen in the treatment of the gum condition in the individual.
